(19) United States
(12) Patent Application Publication
(10) Pub. No.: US 2011/0307977 A1
(43) Pub. Date: Dec. 15, 2011

(54) Grass Seed Providing Distinguishing Color From Surrounding Environments

(75) Inventor: Glen S. AXELROD, Colts Neck, NJ (US)

(73) Assignee: T.F.H. PUBLICATIONS, INC., Neptune City, NJ (US)

(21) Appl. No.: 12/797,435

(22) Filed: Jun. 9, 2010

(51) Int. Cl. A01G 1/00; A01H 5/10

(52) U.S. Cl. 800/298; 47/58.1 R

(57) ABSTRACT

This disclosure relates to seed as may be employed to grow a lawn and more particularly, to grass seed which is imparted with a color which allows it to be relatively easily observed and distinguished within the environment in which it may be distributed. The user may then confirm, among other things, the presence of absence of seed distribution and the uniformity or non-uniformity of any such distribution on a given surface. The color may also promote germination and the colorant may include other additives to improve growth patterns at desired locations.

### FIELD

This disclosure relates to seed as may be employed to grow a lawn and more particularly, to grass seed which is imparted with a color which allows it to be relatively easily observed and distinguished within the environment in which it may be distributed. The user may then confirm, among other things, the presence of absence of seed distribution and the uniformity or non-uniformity of any such distribution on a given surface. The color may also promote germination and the colorant may include other additives to improve growth patterns at desired locations.

### BACKGROUND

Traditionally, seed for growing a lawn or a widespread crop, such as wheat, hay, rye and the like, is broadcast relatively uniformly over a wide area to promote relatively even distribution of the crop and relatively even access of the individual blades or sprigs to water and sunlight. In the case of a lawn, it may be desirable to provide a relatively uniform distribution of the blades to provide a relatively uniform appearance of the coverage of the grass, particularly when freshly mowed.

A common method of starting grass growth on land is to broadcast the seeds directly onto the soil, which may first be prepared by plowing to loosen the soil, or seeds may be broadcast directly onto the soil followed by raking to disturb the top surface of the soil and at least partially cover the seeds.

One problem that may occur is when the seed has a color that closely resembles or blends with the color attributes of the surrounding soil, etc. it may become difficult to determine whether the seed has been uniformly distributed in order to achieve the desired result of uniform growth.

Further, another problem, particularly with grass seed is that most varieties must be planted at or near the surface or at very shallow depths below the surface of the soil. The seeds are therefore subject to being washed away by rain, blown away by wind and eaten by birds, insects and rodents. Again, a means of assessing the distribution of the seed over an area for the critical time before the seed may germinate and root would provide a benefit to most seeding procedures.

It may also be beneficial, particularly for repairing bare spots in lawns, or to control overseeding, to color the seeds to aid in visually distinguishing seed placement from the existing growth of grass. Accordingly, once again, the user may visually confirm that the spot has been properly seeded and confirm that the seed remains at such targeted location until the seeds have germinated so that they are indeed retained and allowed to root.

### SUMMARY

In a first embodiment, the present disclosure is directed at a method of distinguishing seeds from a surrounding environment, comprising providing a sample of soil surface for coverage of vegetation, the surface determined to have a first PMS color number. This may then be followed by providing seed for the vegetation for distribution on the soil surface, wherein the seed is selected to have a second PMS color number wherein the second PMS color number is at least +/−1 PMS color numbers different from the first PMS color number.

In a second embodiment, the present disclosure is directed at a method of distinguishing seeds from vegetation, comprising providing a sample of vegetation, the vegetation determined to have a first PMS color number. This may then be followed by providing seed for distribution on a surface proximate the vegetation, wherein the seed is selected to have a second PMS color number wherein the second PMS color number is at least +/−1 PMS color numbers different from the first PMS color number.

In a third embodiment, the present disclosure is directed at seed, which is visually distinguishable from a surface of surrounding soil by the color of said seed, comprising seed which includes a pigment or a dye, the pigmented or dye having a first color, and said surrounding soil surface having a second color, wherein said first and second colors are different from one another according to the Pantone Matching System (PMS) and wherein said colorant provides one or more of the following:

a. increases the amount of absorbed sunlight on said seed relative to seed without said colorant;

b. a hydrophilic component wherein said hydrophilic component attracts moisture uptake of said seed;

c. a fertilizer component; or

d. a pesticide component

### DETAILED DESCRIPTION

Still other objects and advantages of the present disclosure will become readily apparent to those skilled in the art from the following detailed description, wherein it is shown and described preferred embodiments of the invention. As will be realized the invention is capable of other and different embodiments, and its several details are capable of modification in various respects, without departing from the invention. Accordingly, the description is to be regarded as illustrative in nature and not as restrictive.

Seed, particularly grass seed, is not typically colored to distinguish its' appearance and/or growth characteristics from the surface where it may have been distributed. Accordingly, seed generally has a light brown or tan color, or the color of a dried version of the crop being planted (e.g. the color of hay or relatively light tan). Such a light brown or tan color may therefore make the seed indistinguishable from the surrounding soil where it has been spread and increases the difficulty of assessing whether the seed has been, or remains, uniformly distributed.

Coloring seed, particularly with a distinctive color as disclosed herein, now provides a method to assess the relative uniformity of the distribution of the seed across an area and to assess over time whether the distribution remains static or alters with environmental conditions. “Colorant” as used herein refers to any material which may be added to the seed to alter the seed color. The colorant may be a solid pigment on its own and/or it may be insoluble in a liquid vehicle (resulting in a suspension) that may be used to treat the seed, or a dye, which either is itself a liquid or is soluble in a vehicle (resulting in a solution). Accordingly, the present disclosure contemplates both the use of pigments and dyes to induce a change in seed color for the purpose of distinguishing the seed from a given environment in which it may be applied.

Either or both of the pigment or dye may preferably have an affinity to the surface of the seed to which it is being applied. Such affinity may be chemical in the sense that the pigment or dye has a polarity thereby providing a chemical association to the seed surface. It is therefore contemplated that the pigment or dye may be associated by secondary interactions (dipole-dipole) and/or hydrogen bonds and in certain situations, the pigment or dye may be covalently bonded to the seed surface.

The pigment (which may be insoluble in a given liquid vehicle) and/or dye (liquid or dissolved in a given liquid) may therefore be applied as a liquid to the surface of the seed. Preferably, the concentration of the pigment or dye may be at or below about 10% by weight when the pigment or dye is present in a given liquid. In addition, the pigment or dye may be selected such that its function as a colorant is designed to remain for a period of up to and including 30 days after application, which is generally sufficient for the seed to germinate and take root. In that regard, one may now configure a pigment or dye to actually fade due to exposure to sunlight and/or moisture for a period up to and including 30 days, so that the distinguishing color is extinguished and the natural color of the germinating seed then controls the visual impression generated. As may be appreciated, one may then select a pigment or dye that serves to distinguish for 1, 2, 3, 4, 5 days, up to 30 days, where the dye will not serve to distinguish at any one of the selected days of the 30 day time period.

Alternatively, the colorant may preferably be one that is not removed by exposure to moisture or sunlight and provides a requisite permanence to retain its function as a visual indication of seed presence. In this configuration, one may prefer to retain the distinguishing color of the seed for as long a time as possible so that the need for reseeding may be minimized or removed.

As the present disclosure is directed at providing a seed that is visually distinguished from its surrounding environment, preferably with respect to seeding for the development of lawn grass, it is useful to point out that grasses may be generally understood herein as one or more technically graminoids, and as such are monocotyledonous, usually herbaceous plants with relatively narrow leaves or blades growing from the base. They include the “true grasses”, of the Poaceae (or Gramineae) family, as well as the sedges (Cyperaceae) and the rushes (Juncaceae). The true grasses include cereals, bamboo and the grasses of lawns (turf) and grassland. Sedges include many wild marsh and grassland plants, and some cultivated ones such as water chestnut (Eleocharis dulcis) and papyrus sedge (Cyperus papyrus). Uses for graminoids include food (as grain, sprouted grain, shoots or rhizomes), drink (beer, whisky), pasture for livestock, thatch, paper, fuel, insulation, construction, sports turf, basket weaving and many others. “Visually distinguished” or “visually distinguishable” as used herein refers to an area of soil wherein seed spread thereon appears to the eye to be visually different in character from the surrounding soil or the adjacent vegetation. This is discussed more fully herein with respect to the Pantone Matching System (PMS) available from Pantone LLC which is a wholly owned subsidiary of X-Rite Inc., which allows one to quantify the distinguishing color schemes contemplated herein.

Accordingly, the present disclosure is directed at the formation of a grass seed that has a distinguishing color from its surroundings, and preferably, as most grasses have a green color, to provide a grass seed with a particular green color that is different and distinguishable from its surroundings so that it may be visually detected and confirmed as present on a given surface where seeding may be desired.

Preferably, and as described in more detail herein, one may now utilize pigments or dyes that do not adversely influence the seeding performance or the relatively delicate biochemical balance of a given seeding process. In such context, natural pigments and/or dyes are preferred, which may be understood as those dyes that are not chemically synthesized. For example, the present disclosure contemplates that one may utilize chlorophyll, alone or in combination with other materials in the role of either a pigment or dye for grass seed. Accordingly, one may rely principally on chlorophyll and nevertheless adjust the natural color of chlorophyll to provide a seed with a color that is distinguishable from its surroundings when applied to a given soil surface. It should also be noted that chlorophyll is available in both a natural green and/or red color and both forms of such chlorophyll are contemplated for use herein.

It is useful to note that chlorophyll is registered as a food additive (colorant), and has also been assigned an “E number” of E140. E numbers are understood as number codes for food additives that have been assessed for use within the European Union. The E numbers may also be associated with a particular color group. For example, E numbers of 100-199 are associated with various color groups and in such grouping, E numbers from 140-149 are associated with green coloration. E140 is associated with chlorophylls, E141 is associated with copper complexes of chlorophyll, E142 is associated with Green S, and E143 is associated with Fast Green. In any event, the chlorophylls herein that may be used to provide a distinguishing pigment to a given grass seed may now include any one of more of those chlorophylls represented by the following chemical structures: C₅₅H₇₂O₅N₄Mg (Chlorophyll a), C₅₅H₇₀O₆N₄Mg ((Chlorophyll b), C₃₅H₃₀O₅N₄Mg (Chlorophyll c1), C₃₅H₂₈O₅N₄Mg (Chlorophyll c2) and C₅₄H₇₀O₆N₄Mg (Chlorophyll d). One particularly preferred chlorophyll that may be utilized herein is sodium-copper chlorophyllin.

It is now further contemplated that the color applied to seeds to aid in distinguishing them from soil may comprise and be characterized as hues as identified by the Pantone Matching System (PMS) which allows one to characterize specific colors by an associated PMS color number. PMS color numbers may preferably run between Process Yellow C, PMS 100, PMS 101, PMS 102, Pantone Yellow, PMS 103-PMS 125, PMS 1205, PMS 1215, PMS 1225, PMS 1235, PMS 1245, PMS 1255, PMS 1265, PMS 127-PMS 140, PMS 1345, PMS 1355, PMS 1365, PMS 1275, PMS 1385, PMS 1395, PMS 1405, PMS 141-PMS 154, PMS 1485, PMS 1495, PMS 1505, Orange 021, PMS 1525, PMS 1535, PMS 1545, PMS 155-PMS 161, PMS 1555, PMS 1565, PMS 1575, PMS 1585, PMS 1595, PMS 1605, PMS 1615, PMS 162-PMS 168, PMS 1625, PMS 1635, PMS 1645, PMS 1655, PMS 1685, PMS 169-PMS 178, Warm Red, PMS 179-PMS 181, PMS 1765, PMS 1775, PMS 1785, PMS 1788, PMS 1795, PMS 1805, PMS 1815, PMS 1767, PMS 1777, PMS 1787, Red 032, PEM 1797, PMS 1807, PMS 1817, PMS 182-PMS 195, PMS 1895, PMS 1905, PMS 1915, PMS 1925, PMS 1935, PMS 1945, PMS 1955, PMS 196-PMS 219, Rubine Red, PMS 220-PMS 232, Rhodaimine Red, PMS 233-PMS 242, PMS 2365, PMS 2375, PMS 2385, PMS 2395, PMS 2405, PMS 2415, PMS 2425, PMS 243-PMS 252, Purple, PMS 253-PMS 262, PMS 2562, PMS 2572, PMS 2582, PMS 2592, PMS 2602, PMS 2612, PMS 2622, PMS 2563, PMS 2573, PMS 2583, PMS 2593, PMS 2603, PMS 2613, PMS 2567, PMS 2577, PMS 2587, PMS 2597, PMS 2607, PMS 2617, PMS 2627, PMS 263-PMS 269, PMS 2635, PMS 2645, PMS 2655, PMS 2665, Violet, PMS 2685, PMS 2695, PMS 270-PMS 276, PMS 2705, PMS 2715, PMS 2725, PMS 2735, PMS 2745, PMS 2755, PMS 2765, PMS 2706, PMS 2716, PMS 2726, PMS 2736, PMS 2746, PMS 2756, PMS 2766, PMS 2707, PMS 2717, PMS 2727, Blue 072, PMS 2747, PMS 2757, PMS 2767, PMS 2708, PMS 2718, PMS 2728, PMS 2738, PMS 2748, PMS 2758, PMS 2768, PMS 277-PMS 279, Reflex Blue, PMS 280-PMS 296, PMS 2905, PMS 2915, PMS 2925, PMS 2935, PMS 2945, PMS 2955, PMS 2965, PMS 297-PMS 303, PMS 2975, PMS 2985, PMS 2995, PMS 3005, PMS 3015, PMS 3025, PMS 3035, PMS 304-PMS 306, Process Blue, PMS 307-PMS 316, PMS 3105, PMS 3115, PMS 3125, PMS 3135, PMS 3145, PMS 3155, PMS 3165, PMS 317-PMS 330, PMS 3242, PMS 3252, PMS 3262, PMS 3272, PMS 3282, PMS 3292, PMS 3302, PMS 3245, PMS 3255, PMS 3265, PMS 3275, PMS 3285, PMS 3295, PMS 3305, PMS 3248, PMS 3258, PMS 3268, PMS 3278, PMS 3288, PMS 3298, PMS 3308, PMS 331-PMS 333, Green, PMS 334-PMS 343, PMS 3375, PMS 3385, PMS 3395, PMS 3405, PMS 3415, PMS 3425, PMS 3435, PMS 344-PMS 399, PMS 3935, PMS 3945, PMS 3955, PMS 3965, PMS 3975, PMS 3985, PMS 3995, PMS 400-PMS 405, Black, PMS 406-PMS 447, Warm Gray 1 to Warm Gray 11, Cool Gray 1 to Cool Gray 11, PMS 448-PMS 454, PMS 4485, PMS 4495, PMS 4505, PMS 4515, PMS 4525, PMS 4535, PMS 4545, PMS 455-PMS 468, PMS 4625, PMS 4635, PMS 4645, PMS 4655, PMS 4665, PMS 4675, PMS 4685, PMS 469, PMS 4695, PMS 4705, PMS 4715, PMS 4725, PMS 4735, PMS 4745, PMS 4755, PMS 476-PMS 503, PMS 4975, PMS 4985, PMS 4995, PMS 5505, PMS 5015, PMS 5025, PMS 5035, PMS 504-PMS 517, PMS 5115, PMS 5125, PMS 5135, PMS 5145, PMS 5155, PMS 5165, PMS 5175, PMS 518-PMS 524, PMS 5185, PMS 5195, PMS 5205, PMS 5215, PMS 5225, PMS 5235, PMS 5245, PMS 525-531, PMS 5255, PMS 5265, PMS 5275, PMS 5285, PMS 5295, PMS 5305, PMS 5315, PMS 532-PMS 545, PMS 5395, PMS 5405, PMS 5415, PMS 5425, PMS 5435, PMS 5445, PMS 5455, PMS 546-552, PMS 5463, PMS 5473, PMS 5483, PMS 5493, PMS 5503, PMS 5513, PMS 5423, PMS 5467, PMS 5477, PMS 5487, PMS 5497, PMS 5507, PMS 5517, PMS 5527, PMS 553-559, PMS 5535, PMS 5545, PMS 5555, PMS 5565, PMS 5575, PMS 5585, PMS 5595, PMS 560-566, PMS 5605, PMS 5615, PMS 5625, PMS 5635, PMS 5645, PMS 5655, PMS 568-580, PMS 5743, PMS 5753, PMS 5763, PMS 5773, PMS 5783, PMS 5793, PMS 803, PMS 5747, PMS 5757, PMS 5767, PMS 5777, PMS 5787, PMS 5797, PMS 5807, PMS 581-587, PMS 5815, PMS 5825, PMS 5235, PMS 5845, PMS 5855, PMS 5865, PMS 5875, PMS 600-PMS 732.

Accordingly, in the context of the present disclosure, when the surrounding surface of soil may be associated with a particular PMS color number, the seed for distribution on such surface may be selected so that it has a PMS color number that is at least +/−1 PMS color number removed from the particular PMS color number for the surroundings. More preferably, the PMS color number for the seed may be selected such that it has a PMS color number that is at least +/−2 PMS color numbers removed from the particular PMS color number for the surroundings. In yet another preferred embodiment, the PMS color numbers for the seed may be +/−3 PMS color numbers removed from the particular PMS color number for the surroundings. The PMS color numbers for the seed may also be +/−4 PMS color numbers removed from the particular PMS color number for the surroundings or even +/−5 PMS color numbers removed from the particular PMS color number for the surroundings.

The PMS color numbers for the seed may also be +/−6 PMS color numbers removed from the particular PMS color number for the surroundings or even +/−7 PMS color numbers removed from the particular PMS color number for the surroundings. The PMS color numbers for the seed may also be +/−8 PMS color numbers removed from the particular PMS color number for the surroundings or even +/−9 PMS color numbers removed from the particular PMS color number for the surroundings. The PMS color numbers for the seed may also be +/−10 PMS color numbers removed from the particular PMS color number for the surroundings or even +/−11 PMS color numbers removed from the particular PMS color number for the surroundings.

The PMS color numbers for the seed may also be +/−12 PMS color numbers removed from the particular PMS color number for the surroundings or even +/−13 PMS color numbers removed from the particular PMS color number for the surroundings. The PMS color numbers for the seed may also be +/−14 PMS color numbers removed from the particular PMS color number for the surroundings or even +/−15 PMS color numbers removed from the particular PMS color number for the surroundings. The PMS color numbers for the seed may also be +/−16 PMS color numbers removed from the particular PMS color number for the surroundings or even +/−17 PMS color numbers removed from the particular PMS color number for the surroundings.

The PMS color numbers for the seed may also be +/−18 PMS color numbers removed from the particular PMS color number for the surroundings or even +/−19 PMS color numbers removed from the particular PMS color number for the surroundings. The PMS color numbers for the seed may also be +/−20 PMS color numbers removed from the particular PMS color number for the surroundings or even +/−21 PMS color numbers removed from the particular PMS color number for the surroundings. The PMS color numbers for the seed may also be +/−22 PMS color numbers removed from the particular PMS color number for the surroundings or even +/−23 PMS color numbers removed from the particular PMS color number for the surroundings.

The PMS color numbers for the seed may also be +/−24 PMS color numbers removed from the particular PMS color number for the surroundings or even +/−25 PMS color numbers removed from the particular PMS color number for the surroundings. The PMS color numbers for the seed may also be +/−26 PMS color numbers removed from the particular PMS color number for the surroundings or even +/−27 PMS color numbers removed from the particular PMS color number for the surroundings. The PMS color numbers for the seed may also be +/−28 PMS color numbers removed from the particular PMS color number for the surroundings or even +/−29 PMS color numbers removed from the particular PMS color number for the surroundings.

The PMS color numbers for the seed may also be +/−30 PMS color numbers removed from the particular PMS color number for the surroundings or even +/−31 PMS color numbers removed from the particular PMS color number for the surroundings. The PMS color numbers for the seed may also be +/−32 PMS color numbers removed from the particular PMS color number for the surroundings or even +/−33 PMS color numbers removed from the particular PMS color number for the surroundings. The PMS color numbers for the seed may also be +/−34 PMS color numbers removed from the particular PMS color number for the surroundings or even +/−35 PMS color numbers removed from the particular PMS color number for the surroundings.

The PMS color numbers for the seed may also be +/−36 PMS color numbers removed from the particular PMS color number for the surroundings or even +/−37 PMS color numbers removed from the particular PMS color number for the surroundings. The PMS color numbers for the seed may also be +/−38 PMS color numbers removed from the particular PMS color number for the surroundings or even +/−39 PMS color numbers removed from the particular PMS color number for the surroundings. The PMS color numbers for the seed may also be +/−40 PMS color numbers removed from the particular PMS color number for the surroundings or even +/−41 PMS color numbers removed from the particular PMS color number for the surroundings.

The PMS color numbers for the seed may also be +/−42 PMS color numbers removed from the particular PMS color number for the surroundings or even +/−43 PMS color numbers removed from the particular PMS color number for the surroundings. The PMS color numbers for the seed may also be +/−44 PMS color numbers removed from the particular PMS color number for the surroundings or even +/−45 PMS color numbers removed from the particular PMS color number for the surroundings. The PMS color numbers for the seed may also be +/−46 PMS color numbers removed from the particular PMS color number for the surroundings or even +/−47 PMS color numbers removed from the particular PMS color number for the surroundings.

The PMS color numbers for the seed may also be +/−48 PMS color numbers removed from the particular PMS color number for the surroundings or even +/−49 PMS color numbers removed from the particular PMS color number for the surroundings. The PMS color numbers for the seed may also be +/−50 PMS color numbers removed from the particular PMS color number for the surroundings.

As may therefore be appreciated, the larger the PMS color number difference between the seed and surrounding surface (which may be between +/−1 PMS color numbers to +/−50 PMS color numbers), the greater the visual distinction between the seed and surroundings and a relatively more significant visual distinction may be realized.

Preferably, it may be appreciated that in the case of a new seeding procedure, the surroundings will be soil that will generally take on a brown or tan hue. In that regard, the PMS color number for the soil may be PMS 126 and the seed may assume a PMS color number of 125 or 127. The PMS color number for the soil may also assume a color number of PMS 133 and the seed may then assume a color number of PMS 132 or PMS 134. In other situations when confronted with a relatively tan hue such as PMS 139, the seed may assume a color number of PMS 138 or PMS 140. Consistent with such approach, when the soil has a PMS color number of 146, the seed may assume a color number of PMS 145 or PMS 147. In those situations where the soil may contain a portion of a red hue, the PMS color number of the soil may be 159 and the color number of the seed may be PMS 158 or PMS 160.

In the context of the present invention, it may be appreciated that the present invention provides the ability of a homeowner, for example, to provide to a service facility a sample of the surrounding soil for which seeding may be desired. A color of the surrounding soil may then be determined and a seed may then be provided where the distinguishing color is selected according to the general selection criterion noted herein. In such manner, the homeowner may decide for a greater or reduced contrast for the seed, but nonetheless ensuring that the seed provides a visual confirmation of its presence on the soil once distributed. Furthermore, the homeowner may now indicate a desired time period for the distinguishing color to be present, opting, as noted above, for a color distinction to last for a period of between 1-30 days after application and at any period increment which may be influenced by the prevailing weather, especially temperature and precipitation. In such regard, the selected PMS color for the seed may be applied to the seed and programmed to provide a distinguishing color for that time period required by the homeowner.

In those situations where a patch may be applied to an existing lawn, once again, the seed may be colored so that it can be visually detected to confirm that the seed has been properly distributed on the area which requires patching. For example, for a patch within an existing lawn that has a PMS color number of 355, the seed may assume a PMS color number of 354 or PMS 356. Similarly, in a lawn environment where the lawn may be assigned a PMS color number of 361, the seed may now assume a PMS color number of 360 or 362.

It should also be appreciated that with respect to the above examples describing green seed selection for either a new lawn to be developed or a patching situation, the PMS color numbers may differ from the underlying soil or surrounding lawn color by +/−1 PMS color number, +/−2 PMS color numbers, +/−3 PMS color numbers, +/−4 PMS color numbers and/or +/−5 PMS color numbers, up to +/−50 PMS color numbers.

One green dye that may be used is Brilliant Green (otherwise known as Malachite green G, Emerald green, Solid green JJO, Diamond green G, Aniline green, Benzaldehyde green, Fast green J) which is a triarylmethane dye having the formula C₂₇H₃₄O₄N₂S. Its CAS number is 633-03-4 and it is soluble in water and alcohol.

Other green dyes which may be used include, but are not limited to, ethyl green, methyl green, Light Green SF yellowish, Light Green, Acid Green, Lissamine green SF, Acid Green 5, Food Green 2, FD&C Green no. 2, Green No. 205, Acid Brilliant Green 5, Pencil Green SF, Oregon Green, Tokyo Green, SNAFL, carboxynaphthofluorescein, Alexa 488 and Dylight 488.

More particularly, brighter green colors are preferred, such as Bright Green which has a hex triplet of #66FF00, Chartruese #7FFF00, Harlequin #3FFF00, Green 009F6B, Electric Green #08FF00, Kelly Green #4CBB17, Lime Green # BFFF00, Pigment Green #00A550, Shamrock Green #009E60 and Spring Green #00FF7F. A hex triplet is a six-digit, three-byte hexadecimal number used to represent colors. It is further contemplated that such green colors may be described by their equivalent RGB values (red, green, blue), HSV (hue, saturation, value) or HSB (hue, saturation, brightness) color space values, for instance, Bright Green #66FF00 has a R value of 102, a G value of 255, a B value of 0 as well as a hue of 96°, a saturation of 100% and a value of 100%.

The seed herein may be conveniently colored by coating the outer surface of the seed with a pigment suspension or dye solution and then drying. The solution or suspension may be applied by spraying, dipping, and other coating methods known in the art.

If relatively darker shade colors (red, green, blue, for instance) are used as the colorant for the seed as compared to a natural tan or light brown, an improvement may be seen in the germination process as the darker color may absorb more heat from sunlight than a lighter color seed (tan, brown) and expedite germination. Accordingly, the present disclosure contemplates not only the use of visually distinguished seeds, but seeds that are also accelerated in their germination performance due to a colorizing effect that increased the amount of absorbed sunlight. Expanding on this concept, it may be appreciated that grass seed germinates quickly when ground temperature is 18° C. (65° F.), but it will take off, albeit more slowly, when the soil is only 13° C. or 15° C. (55° F. to 59° F.). The present disclosure therefore contemplates the use of a colorant that allows for the seed to absorb sunlight and more readily assume a temperature of 18° C. to improve the germination process.

Further, if the colorants are hydrophilic in nature, that is, having a strong affinity for moisture, the germination process may again be improved. Hydrophillic properties of pigments may be improved, for instance, by coating the pigment with a hydrophilic compound, preferably at a level of 1-5 wt. %. For example the hydrophilic compound may include a hydrogel, which may be understood as a polymeric material that is crosslinked but which itself is capable of absorbing up to 50% by weight moisture. Other hydrophilic compounds may also include polyalkylene oxides, such as HO-[OR]ₙ-OH where the value of n is between 1-1000 and R is an alkylene group such as a methylene group (—CH₂) or an ethylene group (—CH₂CH₂—) or even propylene (—CH(CH₃)CH₂). Other hydrophilic polymers may include ₂HN-[OR]ₙ-NH₂ wherein again, the value of n is between 1-1000 and R is as stated above. Accordingly, a colorant or carrier for a colorant that has been treated with a hydrophilic additive may absorb relatively more moisture from the air than a seed that has not been treated and again provide expedited germination.

In addition, the colorant may be applied with selected amounts of an organic and/or inorganic and/or synthetic fertilizers, with instant or time-release characteristics. Pesticides may also be included in the colorant, which includes biological and chemical herbicides, insecticides and fungicides. Accordingly, the colorants noted herein may include 0.1-10% by weight of such additives which will then be directly present on the seed surface. By including such additives in the colorant, a more complete and robust seed may be provided, which may then reduce the amount of fertilizer applications that may be necessary to provide comprehensive lawn development.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A method of distinguishing seeds from a surrounding environment, comprising:
providing a sample of soil surface for coverage of vegetation, said soil surface determined to have a first PMS color number;
providing seed for said vegetation for distribution on said surface, wherein said seed is selected to have a second PMS color number wherein said second PMS color number is at least +/−1 PMS color numbers different from said first PMS color number.

2. The method of claim 1 wherein said second PMS color number is at least +/−2 color numbers different from said first PMS color number.

3. The method of claim 1 wherein said second PMS color number is at least +/−3 color numbers different from said first PMS color number.

4. The method of claim 1 wherein said second PMS color number is at least +/−4 color numbers different from said first PMS color number.

5. The method of claim 1 wherein said second PMS color number is at least +/−5 color numbers different from said first PMS color number.

6. The method of claim 1 wherein said second PMS color number is selected from one of the following PMS color numbers: Process Yellow C, PMS 100, PMS 101, PMS 102, Pantone Yellow, PMS 103-PMS 125, PMS 1205, PMS 1215, PMS 1225, PMS 1235, PMS 1245, PMS 1255, PMS 1265, PMS 127-PMS 140, PMS 1345, PMS 1355, PMS 1365, PMS 1275, PMS 1385, PMS 1395, PMS 1405, PMS 141-PMS 154, PMS 1485, PMS 1495, PMS 1505, Orange 021, PMS 1525, PMS 1535, PMS 1545, PMS 155-PMS 161, PMS 1555, PMS 1565, PMS 1575, PMS 1585, PMS 1595, PMS 1605, PMS 1615, PMS 162-PMS 168, PMS 1625, PMS 1635, PMS 1645, PMS 1655, PMS 1685, PMS 169-PMS 178, Warm Red, PMS 179-PMS 181, PMS 1765, PMS 1775, PMS 1785, PMS 1788, PMS 1795, PMS 1805, PMS 1815, PMS 1767, PMS 1777, PMS 1787, Red 032, PEM 1797, PMS 1807, PMS 1817, PMS 182-PMS 195, PMS 1895, PMS 1905, PMS 1915, PMS 1925, PMS 1935, PMS 1945, PMS 1955, PMS 196-PMS 219, Rubine Red, PMS 220-PMS 232, Rhodaimine Red, PMS 233-PMS 242, PMS 2365, PMS 2375, PMS 2385, PMS 2395, PMS 2405, PMS 2415, PMS 2425, PMS 243-PMS 252, Purple, PMS 253-PMS 262, PMS 2562, PMS 2572, PMS 2582, PMS 2592, PMS 2602, PMS 2612, PMS 2622, PMS 2563, PMS 2573, PMS 2583, PMS 2593, PMS 2603, PMS 2613, PMS 2567, PMS 2577, PMS 2587, PMS 2597, PMS 2607, PMS 2617, PMS 2627, PMS 263-PMS 269, PMS 2635, PMS 2645, PMS 2655, PMS 2665, Violet, PMS 2685, PMS 2695, PMS 270-PMS 276, PMS 2705, PMS 2715, PMS 2725, PMS 2735, PMS 2745, PMS 2755, PMS 2765, PMS 2706, PMS 2716, PMS 2726, PMS 2736, PMS 2746, PMS 2756, PMS 2766, PMS 2707, PMS 2717, PMS 2727, Blue 072, PMS 2747, PMS 2757, PMS 2767, PMS 2708, PMS 2718, PMS 2728, PMS 2738, PMS 2748, PMS 2758, PMS 2768, PMS 277-PMS 279, Reflex Blue, PMS 280-PMS 296, PMS 2905, PMS 2915, PMS 2925, PMS 2935, PMS 2945, PMS 2955, PMS 2965, PMS 297 -PMS 303, PMS 2975, PMS 2985, PMS 2995, PMS 3005, PMS 3015, PMS 3025, PMS 3035, PMS 304-PMS 306, Process Blue, PMS 307-PMS 316, PMS 3105, PMS 3115, PMS 3125, PMS 3135, PMS 3145, PMS 3155, PMS 3165, PMS 317-PMS 330, PMS 3242, PMS 3252, PMS 3262, PMS 3272, PMS 3282, PMS 3292, PMS 3302, PMS 3245, PMS 3255, PMS 3265, PMS 3275, PMS 3285, PMS 3295, PMS 3305, PMS 3248, PMS 3258, PMS 3268, PMS 3278, PMS 3288, PMS 3298, PMS 3308, PMS 331-PMS 333, Green, PMS 334-PMS 343, PMS 3375, PMS 3385, PMS 3395, PMS 3405, PMS 3415, PMS 3425, PMS 3435, PMS 344-PMS 399, PMS 3935, PMS 3945, PMS 3955, PMS 3965, PMS 3975, PMS 3985, PMS 3995, PMS 400-PMS 405, Black, PMS 406-PMS 447, Warm Gray 1 to Warm Gray 11, Cool Gray 1 to Cool Gray 11, PMS 448-PMS 454, PMS 4485, PMS 4495, PMS 4505, PMS 4515, PMS 4525, PMS 4535, PMS 4545, PMS 455-PMS 468, PMS 4625, PMS 4635, PMS 4645, PMS 4655, PMS 4665, PMS 4675, PMS 4685, PMS 469, PMS 4695, PMS 4705, PMS 4715, PMS 4725, PMS 4735, PMS 4745, PMS 4755, PMS 476-PMS 503, PMS 4975, PMS 4985, PMS 4995, PMS 5505, PMS 5015, PMS 5025, PMS 5035, PMS 504-PMS 517, PMS 5115, PMS 5125, PMS 5135, PMS 5145, PMS 5155, PMS 5165, PMS 5175, PMS 518-PMS 524, PMS 5185, PMS 5195, PMS 5205, PMS 5215, PMS 5225, PMS 5235, PMS 5245, PMS 525-531, PMS 5255, PMS 5265, PMS 5275, PMS 5285, PMS 5295, PMS 5305, PMS 5315, PMS 532-PMS 545, PMS 5395, PMS 5405, PMS 5415, PMS 5425, PMS 5435, PMS 5445, PMS 5455, PMS 546-552, PMS 5463, PMS 5473, PMS 5483, PMS 5493, PMS 5503, PMS 5513, PMS 5423, PMS 5467, PMS 5477, PMS 5487, PMS 5497, PMS 5507, PMS 5517, PMS 5527, PMS 553-559, PMS 5535, PMS 5545, PMS 5555, PMS 5565, PMS 5575, PMS 5585, PMS 5595, PMS 560-566, PMS 5605, PMS 5615, PMS 5625, PMS 5635, PMS 5645, PMS 5655, PMS 568-580, PMS 5743, PMS 5753, PMS 5763, PMS 5773, PMS 5783, PMS 5793, PMS 803, PMS 5747, PMS 5757, PMS 5767, PMS 5777, PMS 5787, PMS 5797, PMS 5807, PMS 581-587, PMS 5815, PMS 5825, PMS 5235, PMS 5845, PMS 5855, PMS 5865, PMS 5875, or PMS 600-PMS 732.

7. The method of claim 1 wherein the second PMS color for said seed is selected to be visually present for a time period between 1-30 days after application.

8. The method of claim 1 wherein the second PMS color for the seed is selected to increase the absorption of heat from sunlight relative to the seed in the absence of said second PMS color.

9. The method of claim 1 wherein the second PMS color for the seed is selected to increase the absorption of moisture of the seed relative to the seed in the absence of said second PMS color.

10. The method of claim 9 wherein a hydrophilic additive is added to said second PMS color.

11. The method of claim 10 wherein said hydrophilic additive is present at a level of 1.0 to 5.0 wt. %.

12. The method of claim 1 wherein said seed is grass seed.

13. The method of claim 1 wherein said second color is formed by applying a pigment or a dye to said seed.

14. The method of claim 13 wherein said pigment comprises chlorophyll.

15. A method of distinguishing seeds from vegetation, comprising:
providing a sample of vegetation, said vegetation determined to have a first PMS color number;
providing seed for distribution on a surface proximate said vegetation, wherein said seed is selected to have a second PMS color number wherein said second PMS color number is at least +/−1 PMS color numbers different from said first PMS color number.

16. The method of claim 15 wherein said second PMS color number is at least +/−2 color numbers different from said first PMS color number.

17. The method of claim 15 wherein said second PMS color number is at least +/−3 color numbers different from said first PMS color number.

18. The method of claim 15 wherein said second PMS color number is at least +/−4 color numbers different from said first PMS color number.

19. The method of claim 15 wherein said second PMS color number is at least +/−5 color numbers different from said first PMS color number.

20. The method of claim 15 wherein said second PMS color number is selected from one of the following PMS color numbers: Process Yellow C, PMS 100, PMS 101, PMS 102, Pantone Yellow, PMS 103-PMS 125, PMS 1205, PMS 1215, PMS 1225, PMS 1235, PMS 1245, PMS 1255, PMS 1265, PMS 127-PMS 140, PMS 1345, PMS 1355, PMS 1365, PMS 1275, PMS 1385, PMS 1395, PMS 1405, PMS 141-PMS 154, PMS 1485, PMS 1495, PMS 1505, Orange 021, PMS 1525, PMS 1535, PMS 1545, PMS 155-PMS 161, PMS 1555, PMS 1565, PMS 1575, PMS 1585, PMS 1595, PMS 1605, PMS 1615, PMS 162-PMS 168, PMS 1625, PMS 1635, PMS 1645, PMS 1655, PMS 1685, PMS 169-PMS 178, Warm Red, PMS 179-PMS 181, PMS 1765, PMS 1775, PMS 1785, PMS 1788, PMS 1795, PMS 1805, PMS 1815, PMS 1767, PMS 1777, PMS 1787, Red 032, PEM 1797, PMS 1807, PMS 1817, PMS 182-PMS 195, PMS 1895, PMS 1905, PMS 1915, PMS 1925, PMS 1935, PMS 1945, PMS 1955, PMS 196-PMS 219, Rubine Red, PMS 220-PMS 232, Rhodaimine Red, PMS 233-PMS 242, PMS 2365, PMS 2375, PMS 2385, PMS 2395, PMS 2405, PMS 2415, PMS 2425, PMS 243-PMS 252, Purple, PMS 253-PMS 262, PMS 2562, PMS 2572, PMS 2582, PMS 2592, PMS 2602, PMS 2612, PMS 2622, PMS 2563, PMS 2573, PMS 2583, PMS 2593, PMS 2603, PMS 2613, PMS 2567, PMS 2577, PMS 2587, PMS 2597, PMS 2607, PMS 2617, PMS 2627, PMS 263-PMS 269, PMS 2635, PMS 2645, PMS 2655, PMS 2665, Violet, PMS 2685, PMS 2695, PMS 270-PMS 276, PMS 2705, PMS 2715, PMS 2725, PMS 2735, PMS 2745, PMS 2755, PMS 2765, PMS 2706, PMS 2716, PMS 2726, PMS 2736, PMS 2746, PMS 2756, PMS 2766, PMS 2707, PMS 2717, PMS 2727, Blue 072, PMS 2747, PMS 2757, PMS 2767, PMS 2708, PMS 2718, PMS 2728, PMS 2738, PMS 2748, PMS 2758, PMS 2768, PMS 277-PMS 279, Reflex Blue, PMS 280-PMS 296, PMS 2905, PMS 2915, PMS 2925, PMS 2935, PMS 2945, PMS 2955, PMS 2965, PMS 297-PMS 303, PMS 2975, PMS 2985, PMS 2995, PMS 3005, PMS 3015, PMS 3025, PMS 3035, PMS 304-PMS 306, Process Blue, PMS 307-PMS 316, PMS 3105, PMS 3115, PMS 3125, PMS 3135, PMS 3145, PMS 3155, PMS 3165, PMS 317-PMS 330, PMS 3242, PMS 3252, PMS 3262, PMS 3272, PMS 3282, PMS 3292, PMS 3302, PMS 3245, PMS 3255, PMS 3265, PMS 3275, PMS 3285, PMS 3295, PMS 3305, PMS 3248, PMS 3258, PMS 3268, PMS 3278, PMS 3288, PMS 3298, PMS 3308, PMS 331-PMS 333, Green, PMS 334-PMS 343, PMS 3375, PMS 3385, PMS 3395, PMS 3405, PMS 3415, PMS 3425, PMS 3435, PMS 344-PMS 399, PMS 3935, PMS 3945, PMS 3955, PMS 3965, PMS 3975, PMS 3985, PMS 3995, PMS 400-PMS 405, Black, PMS 406-PMS 447, Warm Gray 1 to Warm Gray 11, Cool Gray 1 to Cool Gray 11, PMS 448-PMS 454, PMS 4485, PMS 4495, PMS 4505, PMS 4515, PMS 4525, PMS 4535, PMS 4545, PMS 455-PMS 468, PMS 4625, PMS 4635, PMS 4645, PMS 4655, PMS 4665, PMS 4675, PMS 4685, PMS 469, PMS 4695, PMS 4705, PMS 4715, PMS 4725, PMS 4735, PMS 4745, PMS 4755, PMS 476-PMS 503, PMS 4975, PMS 4985, PMS 4995, PMS 5505, PMS 5015, PMS 5025, PMS 5035, PMS 504-PMS 517, PMS 5115, PMS 5125, PMS 5135, PMS 5145, PMS 5155, PMS 5165, PMS 5175, PMS518-PMS 524, PMS 5185, PMS 5195, PMS 5205, PMS 5215, PMS 5225, PMS 5235, PMS 5245, PMS 525-531, PMS 5255, PMS 5265, PMS 5275, PMS 5285, PMS 5295, PMS 5305, PMS 5315, PMS 532-PMS 545, PMS 5395, PMS 5405, PMS 5415, PMS 5425, PMS 5435, PMS 5445, PMS 5455, PMS 546-552, PMS 5463, PMS 5473, PMS 5483, PMS 5493, PMS 5503, PMS 5513, PMS 5423, PMS 5467, PMS 5477, PMS 5487, PMS 5497, PMS 5507, PMS 5517, PMS 5527, PMS 553-559, PMS 5535, PMS 5545, PMS 5555, PMS 5565, PMS 5575, PMS 5585, PMS 5595, PMS 560-566, PMS 5605, PMS 5615, PMS 5625, PMS 5635, PMS 5645, PMS 5655, PMS 568-580, PMS 5743, PMS 5753, PMS 5763, PMS 5773, PMS 5783, PMS 5793, PMS 803, PMS 5747, PMS 5757, PMS 5767, PMS 5777, PMS 5787, PMS 5797, PMS 5807, PMS 581-587, PMS 5815, PMS 5825, PMS 5235, PMS 5845, PMS 5855, PMS 5865, PMS 5875, or PMS 600-PMS 732.

21. The method of claim 15 wherein the second PMS color for said seed is selected to be visually present for a time period between 1-30 days after application.

22. The method of claim 15 wherein the second PMS color for the seed is selected to increase the absorption of heat from sunlight relative to the seed in the absence of said second PMS color.

23. The method of claim 15 wherein the second PMS color for the seed is selected to increase the absorption of moisture of the seed relative to the seed in the absence of said second PMS color.

24. The method of claim 23 wherein a hydrophilic additive is added to said second PMS color.

25. The method of claim 24 wherein said hydrophilic additive is present at a level of 1.0 to 5.0 wt. %.

26. The method of claim 15 wherein said seed is grass seed.

27. The method of claim 15 wherein said second color is formed by applying a pigment or a dye to said seed.

28. The method of claim 15 wherein said pigment comprises chlorophyll.

29. The method of claim 15 wherein said vegetation is grass.

30. Seed, which is visually distinguishable from a surface of surrounding soil by the color of said seed, comprising seed which includes a pigment or a dye, said pigmented or dye having a first color and said surrounding soil surface having a second color, wherein said first and second colors are different from one another according to the Pantone Matching System and wherein said colorant provides one or more of the following:
a. increases the amount of absorbed sunlight on said seed relative to seed without said colorant;
b. a hydrophilic component wherein said hydrophilic component attracts moisture uptake of said seed;
c. a fertilizer component; or
d. a pesticide component.
